(19) United States
(12) Patent Application Publication
(10) Pub. No.: US 2022/0349892 A1
(43) Pub. Date: Nov. 3, 2022

(54) Use of TM9SF4 as a biomarker for tumor associated exosomes

(71) Applicant: Exosomics S.p.A., Siena (IT)

(72) Inventors: Francesco LOZUPONE, Roma (IT); Antonio Chiesi, Vigasio (Verona) (IT); Paolo Guazzi, Tallinn (EE); Natasa Zarovni, Milano (IT); Pietro Ferruzzi, Firenze (IT); Davide Zocco, Siena (IT)

(21) Appl. No.: 17/737,453

(22) Filed: May 5, 2022

Related U.S. Application Data
(63) Continuation of application No. 15/316,188, filed on Dec. 5, 2016, now abandoned.

(30) Foreign Application Priority Data
Jun. 6, 2014 (EP) 14171464.2

(51) Int. Cl. G01N 33/574

(52) U.S. Cl. CPC G01N 33/57492; G01N 33/57488; G01N 33/574; G01N 2800/56

(57) ABSTRACT

The present invention relates to extracellular microvesicles biomarkers for determining the tumour transformation status or presence of a tumour in a subject, and to the uses of such biomarkers and to diagnostics methods using such biomarkers. In particular, the methods and uses of the invention involve isolation of TM9SF4-positive extracellular vesicles and detection of the expression of a second biomarker, preferably selected from the group consisting of CD9 protein, miR-21 and RNU6.

### CROSS REFERENCES

This application is continuation application of U.S. application Ser. No. 15/316,188 filed on Dec. 5, 2016 as a U.S national stage filing of PCT/EP2015/062594 filed on Jun. 5, 2015 the contents of both of which are incorporated herein by reference.

### STATEMENT REGARDING SEQUENCE LISTING

Applicant submits herewith a Sequence Listing in computer readable form and in compliance with 37 C.F.R. §§ 1.821-1.825. This sequence listing is in ASCII TXT format with filename “11353-004US2_2022_05_05_Sequence,” a 8,127 byte file size, and creation date of May 5, 2022. The content of the Sequence Listing is hereby incorporated by reference.

### FIELD OF INVENTION

The present invention relates to extracellular microvesicles biomarkers for determining the tumour transformation status or presence of a tumour in a subject, and to the uses of such biomarkers and to diagnostics methods using such biomarkers.

### BACKGROUND TO THE INVENTION

Contrary to malignant (or cancerous) tumours, benign tumours typically are mass of cells that lack the ability to invade neighbouring tissue or metastasise. Also, benign tumours generally have a slower growth rate than malignant tumours and the tumour cells are usually more differentiated.

Although most benign tumours are not life-threatening, many types of benign tumours have the potential to become cancerous (malignant) through a process known as tumour transformation.

Non-Metastatic Cancer (primary or recurrent) is a cancer that has not spread from the primary site (place where it started) to other places in the body.

Metastatic cancer is a cancer that has spread from the part of the body where it started (the primary site) to other parts of the body.

The development of benign neurofibromas can often be linked to a mutation of the NF1 tumor suppressor gene in cells of the Schwann cell lineage¹⁻³. These neoplasms can frequently undergo a further transformation to malignant peripheral nerve sheet tumors (MPNSTs)¹⁻³. It is currently unclear which cell types are particularly susceptible to MPNST formation, which are the molecular changes causing the development of MPNSTs from neurofibromas, or which other factors in the tumor environment might contribute to neoplasia. In addition, gliomas, particularly pilocytic astrocytomas of the optic nerve, and leukemias, are seen with increased frequency in the NF1 population³.

MPNSTs have very poor prognosis as they do not respond to standard chemo- or radiation therapy and have a high propensity to metastasize⁴⁻⁷. NF1 patients and their families are well aware of these facts, which is why the development of an MPNST is the complication that is most dreaded by patients suffering from this disease⁸. However, early detection is often hampered by the fact that MPNSTs frequently develop within preexisting large neurofibromas, making new growth or progression difficult to detect and distinguish even with MRI. This diagnostic delay is likely the cause of poor outcome of MPNST in NF1 with respect to their sporadic counterparts. This constitutes the major impetus for identification of molecular alterations that can be detected in a noninvasive manner and are indicative of MPNST initiation and progression in NF1 patients that would be useful in screening and early diagnosis as well as monitoring of disease or therapeutic outcome in preclinical and clinical settings.

It is generally agreed that multiple neurofibroma subtypes exist which differ in their location and pattern of growth, their association with NF1 and their potential for malignant transformation. Many clinical and basic science investigators broadly classify neurofibromas as either dermal or plexiform variants¹. Plexiform neurofibromas are neurofibroma variants that occur almost exclusively in NF1 patients and are thought to be congenital; they are distinguished from localized intraneural neurofibromas by their characteristic plexiform growth pattern. Plexiform neurofibromas have the highest risk for malignant transformation into MPNST¹.

Similarly, to neurofibromas transformation into MPNST, other benign tumors have a risk to transform into their malignant counterpart. This is for example the case of Benign Prostatic Hyperplasia (BPH) to prostate cancer, colon polyps to colorectal cancer, benign nevi to melanoma, non-cancerous breast conditions to breast cancer, lung nodules to lung cancer, early-stage astrocytoma to glioblastoma, and benign ovarian tumors to ovarian cancer. Most of these cancers are also able to metastatise

Extracellular vesicles (EVs) are a class of membrane bound organelles secreted by various cell types⁹. EVs not limitedly include (i) exosomes: 30-100 nm diameter membraneous vesicles of endocytic origin (ii) ectosomes (also referred to as shedding microvesicles, SMVs): large membranous vesicles (50-1000 nm diameter) that are shed directly from the plasma membrane (PM) and (iii) apoptotic bodies (50-5000 nm diameter): released by dying cells.

Exosomes are natural lipidic extra cellular nanovescicles produced and released by virtually all cell types in a finely regulated and functionally relevant manner so that the protein and mRNA composition reflects the type and condition of a parent cell¹⁰⁻¹⁴. These vesicles have intrinsic stability and ability to cross biological barriers, so that exosomes originated from different tissues can be found in easily accessible biological fluids such as blood¹⁵⁻¹⁷. Given their biological roles and features, exosomes are considered early sentinels of alterations in cell and tissue homeostasis and metabolism and are an appealing source for identification of novel disease-relevant biomarkers as well as display of known tissue markers in a liquid biopsy paradigm. This is a major premise and promise of using exosome targeted assays in diagnostics of complex diseases such as cancer. The major challenge lies in association of exosome associated markers, both proteins and RNAs, to a particular tissue, in a particular condition and optimization of reliable, affordable, non-invasive exosome targeted solutions and assays that can be realistically implemented in clinical research and practice¹⁸⁻²¹.

There currently is a need for extracellular vesicle biomarkers that are able to determine the presence of a tumour (be it benign, malignant and metastatic) or the transformation status of a tumour (benign to malignant and non-metastatic to metastatic).

### BRIEF DESCRIPTION OF THE FIGURES

FIG. 1 compares the levels of biomarkers TM9SF4 and CD9 measured by FACS on an MPNST cell line (S462, first column), a Plexiform Neurofibroma line (54836T_003, second column) and a dermal neurofibroma cell line (1201A078, third column).

FIG. 2 shows the results of a sandwich Elisa test where 40, 20, 10 and 5 μg of exosomes purified by ultracentrifugation protocol from conditioned media originating from a glioblastoma cell line (U87) or three MPNST cell lines (S462, T265 and 88-14) or from a human embryonic kidney cell line (HEK293) are captured with an anti-TM9SF4 antibody and detected with an anti-CD9 antibody. Ratio to Background reported in the ordinate axis correspond to the absorbance values of each sample divided for the background average absorbance (PBS alone, 0 μg=Ratio to Background 1).

FIG. 3A IHC assessment of TM9SF4 in subjects with Colorectal cancer (CRC) and gastric cancer (GC) compared to healthy surrounding tissue and pre-neoplastic lesions (hyperplastic polyps and tubullovillous adenoma, and gastric dysplasia respectively).

FIG. 3B IHC staining of TM9SF4 positive cells/mm2 in breast, lung and melanoma cancers compared to healthy surrounding tissues. The figure revealed a significant higher number of TM9SF4 positive cells/mm2 in all the cancer tissues analysed.

FIG. 4 shows the results of a sandwich ELISA test where 100 μl of pre-cleared (see materials and methods) plasma samples obtained from early (TNM classification T1-2N0M0) or advanced (TNM classification T3-4NxMx) tumoral stage patients have been immune-captured through TM9SF4 antibody coated 96 well plates. The numbers in the bar-graph corresponded to the number of observations for each study group. Ratio to

Background was calculated by dividing samples absorbance values for the background value (only PBS in the well Ratio to Background=1).

FIG. 5 shows the results of a sandwich ELISA test where 100 μl of pre-cleared (see materials and methods) plasma samples obtained from tumoral patients have been immune-captured through TM9SF4 antibody coated 96 well plates. In the horizontal axis is reported the tumor group and the number of observations (N). Ratio to Background was calculated by dividing samples adsorbance values for the background value (only PBS in the well Ratio to Background=1).

FIG. 6 represents a Receiver Operating Characteristic (ROC) curve calculated by GraphPad Prism program using the Colorectal Cancer (CRC) data reported in FIG. 5. Healthy Donor group was used to calculate the specificity and the optimal threshold of TM9SF4/CD9 ELISA sandwich assay on plasma samples. The figure shows how assuming a threshold of >6.925 the test has a sensitivity >92% and a specificity >95%.

FIG. 7 represents a ROC curve calculated by GraphPad Prism program using the Gastric Cancer data reported in FIG. 5. Healthy Donor group was used to calculate the specificity and the optimal threshold of TM9SF4/CD9 ELISA sandwich assay on plasma samples. The figure shows how assuming a threshold >7.025 the test has a sensitivity >83.9% and a specificity >95%.

FIG. 8 represents a ROC curve calculated by GraphPad Prism program using the Breast Cancer data reported in FIG. 5. Healthy Donor group was used to calculate the specificity and the optimal threshold of TM9SF4/CD9 ELISA sandwich assay on plasma samples. The figure shows how assuming a threshold >7.004 the test has a sensitivity >88.2% and a specificity >95%.

FIG. 9 represents a ROC curve calculated by GraphPad Prism program using the Prostate Cancer data reported in FIG. 5. Healthy Donor group was used to calculate the specificity and the optimal threshold of TM9SF4/CD9 ELISA sandwich assay on plasma samples.

The figure shows how assuming a threshold >7.005 the test has a sensitivity >75.8% and a specificity >95%.

FIG. 10 shows the results of a sandwich ELISA test where 100 μl of pre-cleared SERUM samples obtained from tumoral patients have been immune-captured through TM9SF4 antibody coated 96 well plates.

FIG. 11A shows the results of a sandwich ELISA test where 100 μl of pre-cleared plasma samples obtained from seven colorectal cancer (CRC #1-#7) and control group (healthy donors-HD) have been immune-captured through TM9SF4 antibody coated 96 well plates.

FIG. 11B shows the relative expression of extracellular vesicle-(EV)-derived miR-21 (normalized to miR-451) from 100 μl of the SAME set of samples.

FIG. 12 shows the relative expression of EV-derived miR-21 (normalized to miR-451 or to miR-574) from 100 μl of plasma from cancer patients (Colorectal Cancer (CRC) N=7; Gastric Cancer N=6; Breast Cancer N=6; Prostate Disease N=5; Melanoma N=5; Ovary N=6; Lung Cancer N=6) and control group (healthy donors N=11).

FIG. 13 shows the relative expression of EV-derived RNU6 and EV-derived miR-21 (normalized to miR-223) from 1 ml of concentrated (10×) cell supernatant from dermal, plexiform and MPNST cell lines.

FIG. 14A shows the relative expression of EV-derived miR-21 (normalized to miR-451) from 100 μl of plasma from a prostate cancer patient and a healthy donor.

FIG. 14 B shows the relative expression of EV-derived miR-21 (normalized to miR-451) from 100 μl of serum from a colorectal cancer (CRC) patient and a healthy donor.

FIG. 15 shows the relative expression of EV-derived miR-21 (normalized to miR-451) from 100 μl of plasma from a prostate cancer patient and a healthy donor.

FIG. 16 shows the results of a sandwich ELISA test where 100 μl of pre-cleared (see materials and methods) plasma samples obtained from tumoral patients and healthy donors have been immune-captured through CD9 antibody coated 96 well plates. Ratio to Background was calculated by dividing samples adsorbance values for the background value (only PBS in the well Ratio to Background=1).

### DESCRIPTION OF THE INVENTION

Due to the micellar nature of extracellular vesicles such as exosomes, some biomolecules present in these vesicles can be detected without lysing the vesicles because they reside on the membrane, whereas some others may only be detected after lysis of the vesicles because they are located within the vesicle.

We have surprisingly found that TM9SF4-positive extracellular vesicles (i.e. extracellular vesicles that harbour the TM9SF4 protein) are extremely versatile tools that can be used to determine presence of a tumour or the tumour transformation state in a subject, particularly if a biomarker selected from the list of table 1 is used.

| Biomarker | Type | Detected from |
|---|---|---|
| CD9 | Protein | Extracellular vesicle |
| | | membrane |
| miR-21 | miRNA | Whole Extracellular |
| | | vesicle |
| RNU6 | snRNA | Whole Extracellular |
| | | vesicle |

TM9SF4 protein (SEQ ID NO: 1) is a recently described transmembrane protein that belongs to Transmembrane-9 Superfamily (TM9SF), a well-defined family of proteins characterized by a large hydrophylic N-terminal domain followed by nine transmembrane domains²². This protein is known to be overexpressed in melanoma and in acute myeloid leukemia and myelodysplastic syndromes, latter due to a three to tenfold amplification of a chromosome 20 fragment (20q11.21) bearing the entire TM9SF4 gene23,24. TM9SF4 is involved in phagocytosis of bacteria and in the cannibal phenotype of metastatic melanoma cells, a phenomenon often related with poor prognosis ^{25,26}. Cannibal cancer cells have been frequently detected in gastric and colon cancers²⁷⁻³⁰

It has been recently shown that TM9SF4 binds to V-ATPase, a pH regulating proton pump overexpressed in several tumors. This interaction aberrantly stabilizes the proton pump in its active state with the consequent pH gradient alterations that in turn is associated with drug resistance and invasiveness of colon cancer cells³¹.

CD9 protein (SEQ ID NO: 2) is a member of the transmembrane 4 superfamily, also known as the tetraspanin family. Tetraspanins are cell surface glycoproteins with four transmembrane domains that form multimeric complexes with other cell surface proteins. The encoded protein functions in many cellular processes including differentiation, adhesion, and signal transduction, and expression of this gene plays a critical role in the suppression of cancer cell motility and metastasis. It is found on the surface of exosomes and is considered exosome housekeeping protein for the quantitative analysis of plasma derived nanovesicles.

miRNA21 (SEQ ID NO: 3) miRNAs are a class of small non-coding RNAs whose mature products are ˜22 nucleotides long. They negatively regulate gene expression by inducing translational inhibition or transcript degradation³². miR-21 has been found to be upregulated in many pathological conditions including cancer and cardiovascular diseases³³. The identification of several targets of miRNAs which are actually classical oncogenes or tumor suppressors has led to the widely accepted idea that miRNAs play pivotal roles in cancer initiation, progression and metastasization^{34,35} miR-21 was first noted as an apoptotic suppressor in various cell lines³⁶.

RNU6 (SEQ ID NO: 4) is a non-coding RNA (ncRNA) molecule which functions in the modification of other small nuclear RNAs (snRNAs). Accurate profiling of microRNAs (miRNAs) is an essential step for understanding the functional significance of these small RNAs in both physiological and pathological processes. It is well-known that normalization is one of the most critical steps in qRT-PCR and commonly used genes for this purpose, such as U6 and 5S³⁷, have already been described as being differentially expressed in cancer, which makes these genes not suitable as internal controls.

Accordingly, in a first aspect of this invention, there is provided a method for determining in vitro the presence of a tumour in a subject, such method comprising:

a) providing a biological sample obtained from that subject,

b) isolating extracellular vesicles from said sample, wherein this step of isolating extracellular vesicles comprises isolating TM9SF4-positive extracellular vesicles,

c) determining, from the extracellular vesicles isolated in step b), the level or presence of a suitable biomarker, and

d) comparing the level or presence of the biomarker determined in step c) with one or more reference values.

In one embodiment the subject is suspected of being affected by a tumour.

In one embodiment, the TM9SF4-positive extracellular vesicles are isolated through binding to an anti-TM9SF4 antibody.

In another embodiment, at least a portion of the extracellular vesicles are exosomes.

In a further embodiment, the extracellular vesicles are exosomes.

In one embodiment, the tumour is a malignant tumour.

In one embodiment, the tumour is colon cancer.

In another embodiment, the tumour is gastric cancer.

In another embodiment, the tumour is breast cancer.

In another embodiment, the tumour is lung cancer.

In another embodiment, the tumour is melanoma.

In another embodiment, the tumour is pancreatic cancer.

In another embodiment, the tumour is ovary cancer.

In another embodiment, the tumour is prostate cancer.

In another embodiment the tumour is a central nervous system tumour.

In a particular embodiment, the central nervous system tumour is glioblastoma.

In another embodiment, the tumour is MPNST.

In one embodiment, the biomarker of step c) is CD9 protein.

In another embodiment, the biomarker of step c) is miR-21.

In another embodiment, the biomarker of step c) is RNU6.

In one embodiment, the sample is a tumour sample.

In another embodiment, the sample is a bodily fluid.

In a particular embodiment, the sample is a plasma sample.

In a particular embodiment the sample is a blood sample.

In a particular embodiment the sample is a serum sample.

In a particular embodiment the sample is a urine sample.

In a particular embodiment the sample is a saliva sample.

In one embodiment the subject is a human.

In another embodiment the subject is a mammal.

In one embodiment, the reference value is the level or presence of the same biomarker of step c) in an earlier sample from the same subject as in step a).

In another embodiment, the reference value is the level or presence of the same biomarker of step c) in samples obtained from different subjects than the subject of step a).

Any combination of the above embodiments of this first aspect of the invention represent further embodiments of the invention.

In a second aspect to this invention, there is provided a method for determining in vitro the tumour transformation status in a subject, such method comprising:

a) providing a biological sample obtained from that subject,

b) isolating extracellular vesicles from said sample, wherein this step of isolating extracellular vesicles comprises isolating TM9SF4-positive extracellular vesicles,

c) determining, from the extracellular vesicles isolated in step b), the level or presence of a suitable biomarker, and

d) comparing the level or presence of the biomarker determined in step c) with one or more reference values.

In one embodiment, the biological sample of step a) is obtained from a patient affected by a benign tumour.

In a particular embodiment, the benign tumour is a benign colon tumour.

In a particular embodiment, the benign tumour is a plexiform neurofibroma.

In another embodiment, the TM9SF4-positive extracellular vesicles are isolated through binding to an anti-TM9SF4 antibody.

In another embodiment, at least a portion of the extracellular vesicles are exosomes.

In a further embodiment, the extracellular vesicles are exosomes.

In one embodiment the tumour transformation status is the transformation to an MPNST.

In another embodiment, the tumour transformation status is the transformation to a colorectal cancer.

In one embodiment, the biomarker of step c) is CD9 protein.

In another embodiment, the biomarker of step c) is miR-21.

In another embodiment, the biomarker of step c) is RNU6.

In one embodiment, the sample is a tumour sample.

In another embodiment, the sample is a bodily fluid.

In a particular embodiment, the sample is a plasma sample.

In a particular embodiment the sample is a blood sample.

In a particular embodiment the sample is a serum sample.

In a particular embodiment the sample is a urine sample.

In a particular embodiment the sample is a saliva sample.

In one embodiment the subject is a human.

In another embodiment the subject is a mammal.

In one embodiment, the reference value is the level or presence of the same biomarker of step c) in an earlier sample from the same subject as in step a).

In another embodiment, the reference value is the level or presence of the same biomarker of step c) in samples obtained from different subjects than the subject of step a).

Any combination of the above embodiments of this second aspect of the invention represent further embodiments of the invention.

In a third aspect of this invention, there is provided TM9SF4-positive extracellular vesicles for use in a test to determine the presence of a tumour or the tumour transformation status in a subject.

In one embodiment, the test is an in vitro test.

In one embodiment, the extracellular vesicles are exosomes.

In one embodiment, the tumour is a malignant tumour.

In one embodiment, the tumour is colon cancer.

In another embodiment, the tumour is gastric cancer.

In another embodiment, the tumour is breast cancer.

In another embodiment, the tumour is lung cancer.

In another embodiment, the tumour is melanoma.

In another embodiment, the tumour is pancreatic cancer.

In another embodiment, the tumour is ovary cancer.

In another embodiment, the tumour is prostate cancer.

In another embodiment the tumour is a central nervous system tumour.

In a particular embodiment, the central nervous system tumour is glioblastoma.

In another embodiment, the tumour is MPNST.

In one embodiment the tumour transformation status is the transformation to an MPNST.

In another embodiment, the tumour transformation status is the transformation to a colorectal cancer.

In one embodiment the subject is a human.

In another embodiment the subject is a mammal.

Any combination of the above embodiments of this third aspect of the invention represents further embodiments of the invention.

A fourth aspect of this invention concerns the use of TM9SF4-positive extracellular vesicles in a test to determine the presence of a tumour or the tumour transformation status in a subject.

In one embodiment, the test is an in vitro test

In one embodiment, at least a portion of the extracellular vesicles are exosomes.

In a further embodiment, the extracellular vesicles are exosomes.

In one embodiment, the tumour is a malignant tumour.

In one embodiment, the tumour is colon cancer.

In another embodiment, the tumour is gastric cancer.

In another embodiment, the tumour is breast cancer.

In another embodiment, the tumour is lung cancer.

In another embodiment, the tumour is melanoma.

In another embodiment, the tumour is pancreatic cancer.

In another embodiment, the tumour is ovary cancer.

In another embodiment, the tumour is prostate cancer.

In another embodiment the tumour is a central nervous system tumour.

In a particular embodiment, the central nervous system tumour is glioblastoma.

In another embodiment, the tumour is MPNST.

In one embodiment the tumour transformation status is the transformation to an MPNST.

In another embodiment, the tumour transformation status is the transformation to a colorectal cancer.

In one embodiment the subject is a human.

In another embodiment the subject is a mammal.

Any combination of the above embodiments of this fourth aspect of the invention represents further embodiments of the invention.

In a fifth aspect of this invention, there is provided a kit for use in determining the presence of a tumour or a tumour transformation status in a subject, such kit comprising an anti-TM9SF4 antibody.

In one embodiment, the kit further comprises an anti CD9-antibody.

In another embodiment, the kit further comprises a miR-21 primer.

In another embodiment, the kit further comprises an anti a RNU6 primer.

In one embodiment, the tumour is a malignant tumour.

In one embodiment, the tumour is colon cancer.

In another embodiment, the tumour is gastric cancer.

In another embodiment, the tumour is breast cancer.

In another embodiment, the tumour is lung cancer.

In another embodiment, the tumour is melanoma.

In another embodiment, the tumour is pancreatic cancer.

In another embodiment, the tumour is ovary cancer.

In another embodiment, the tumour is prostate cancer.

In another embodiment the tumour is a central nervous system tumour.

In a particular embodiment, the central nervous system tumour is glioblastoma.

In another embodiment, the tumour is MPNST.

In one embodiment the tumour transformation status is the transformation to an MPNST.

In another embodiment, the tumour transformation status is the transformation to a colorectal cancer.

In another embodiment, the kit further comprises instructions for suitable operational parameters in the form of a label or separate insert.

Any combination of the above embodiments of this fifth aspect of the invention represents further embodiments of the invention.

### EXAMPLES

There now follows by way of example only a detailed description of the present invention with reference to the accompanying drawings, in which:

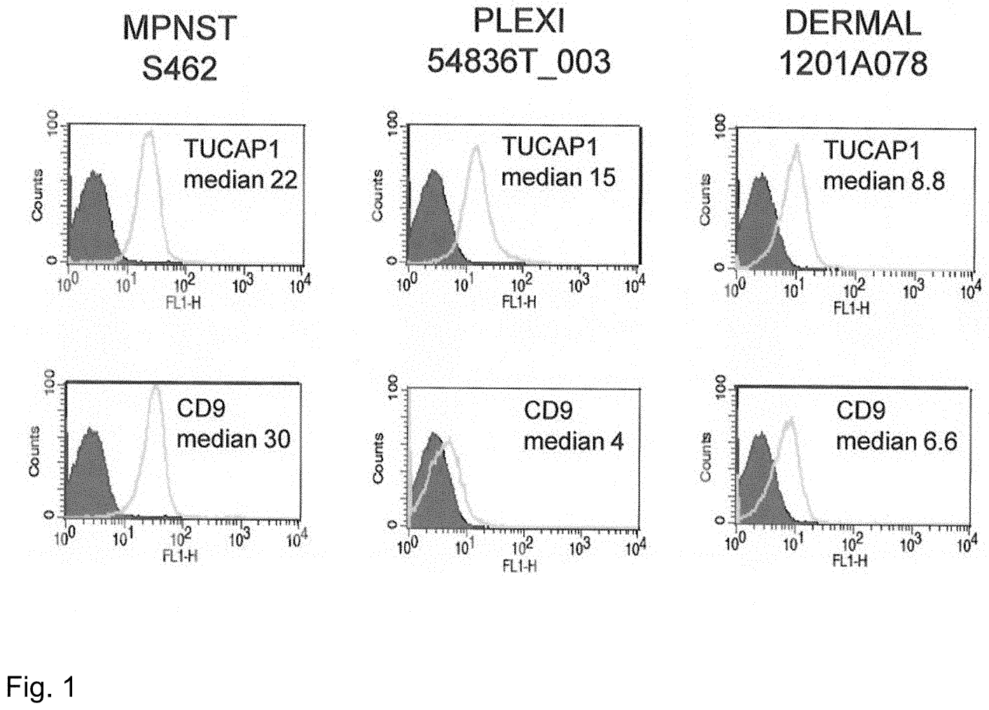

FIG. 1 compares the levels of biomarkers TM9SF4 and CD9 measured by FACS on an MPNST cell line (S462, first column), a Plexiform Neurofibroma line (54836T_003, second column) and a dermal neurofibroma cell line (1201A078, third column). The median values demonstrate that the biomarkers, when detected from the exosome membrane, can differentiate between benign (plexiform neurofibroma, dermal neurofibroma) and malignant (MPNST) conditions.

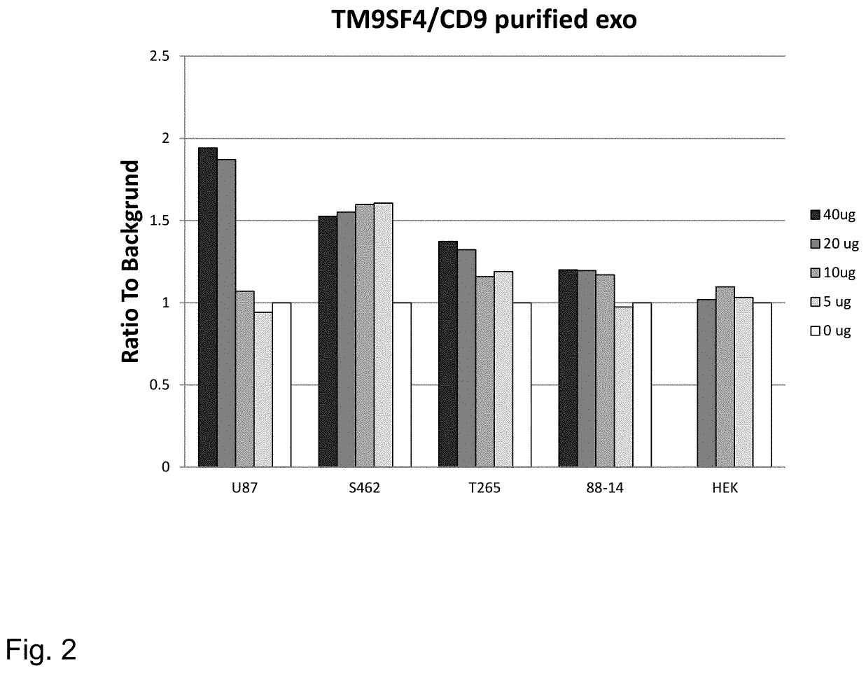

FIG. 2 shows the results of a sandwich Elisa test where 40, 20, 10 and 5 μg of exosomes purified by ultracentrifugation protocol from conditioned media originating from a glioblastoma cell line (U87) or three MPNST cell lines (S462, T265 and 88-14) or from a human embryonic kidney cell line (HEK293) are captured with an anti-TM9SF4 antibody and detected with an anti-CD9 antibody, showing that these biomarkers are expressed on exosomal membrane and that this particular sandwich Elisa assay can be used to detect malignant neurofibroma (MPNST) or other solid tumors (for ex Glioblastoma) derived exosomes and not HEK293 purified exosomes. Ratio to Background reported in the ordinate axis correspond to the absorbance values of each sample divided for the background average absorbance (PBS alone, 0 μg=Ratio to Background 1).

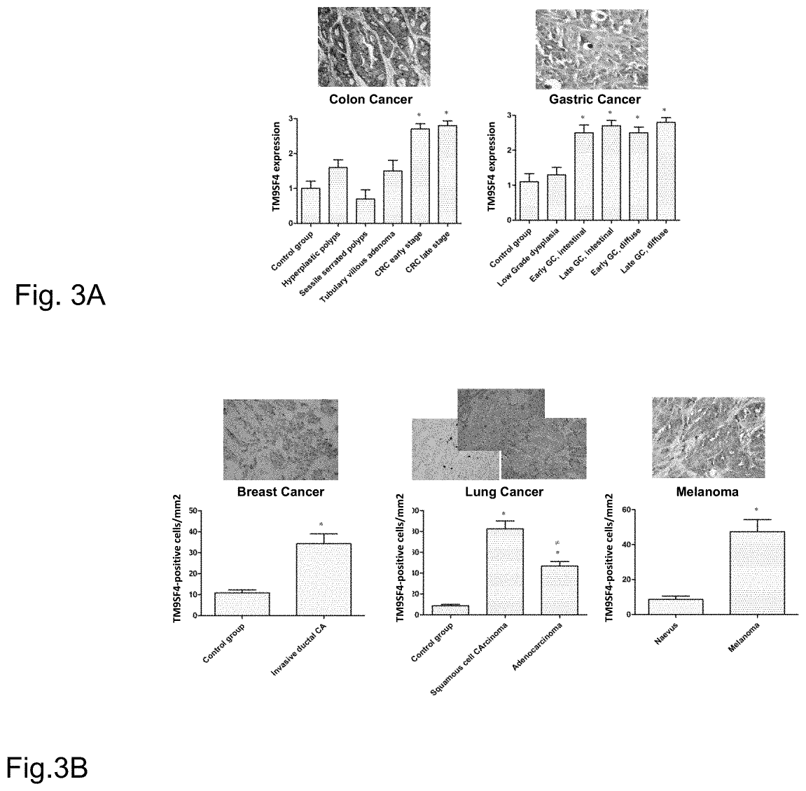

FIG. 3A. IHC assessment of TM9SF4 in subjects with Colorectal cancer (CRC) and gatric cancer(GC) compared to healthy surrounding tissue and pre-neoplastic lesions (hyperplastic polyps and tubullovillous adenoma, and gastric dysplasia respectively), revealed highly specific staining of tumor tissue in both early and advanced stages, with no or little expression in healthy or dysplastic tissue. Overall 90% of cancers examined strongly expressed TM9SF4 and the level of expression (IHC score) significantly correlated with disease stage. FIG. 3B IHC staining of TM9SF4 positive cells/mm2 in breast, lung and melanoma cancers compared to healthy surrounding tissues. The figure revealed a significant higher number of TM9SF4 positive cells/mm2 in all the cancer tissues analysed.

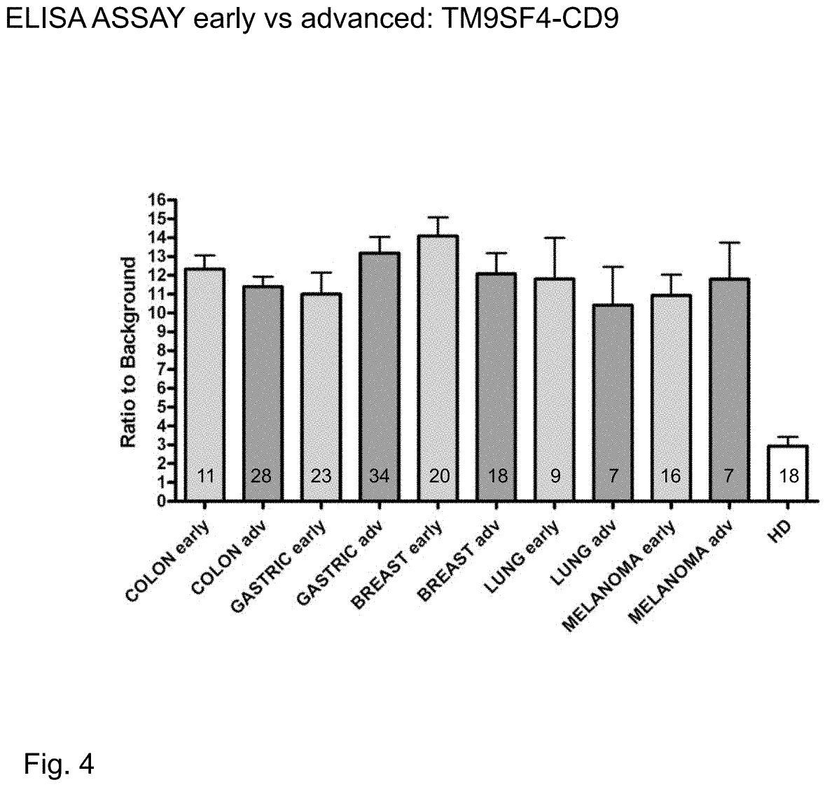

FIG. 4 shows the results of a sandwich ELISA test where 100 μl of pre-cleared (see materials and methods) plasma samples obtained from early (TNM classification T1-2N0M0) or advanced (TNM classification T3-4NxMx) tumoral stage patients have been immune-captured through TM9SF4 antibody coated 96 well plates. The detection by CD9 antibody revealed highly specific Ratio to Background values of tumour plasma samples in both early and advanced stages, with very low expression in healthy donors plasma samples. The numbers in the bar-graph corresponded to the number of observations for each study group. Ratio to Background was calculated by dividing samples absorbance values for the background value (only PBS in the well Ratio to Background=1).

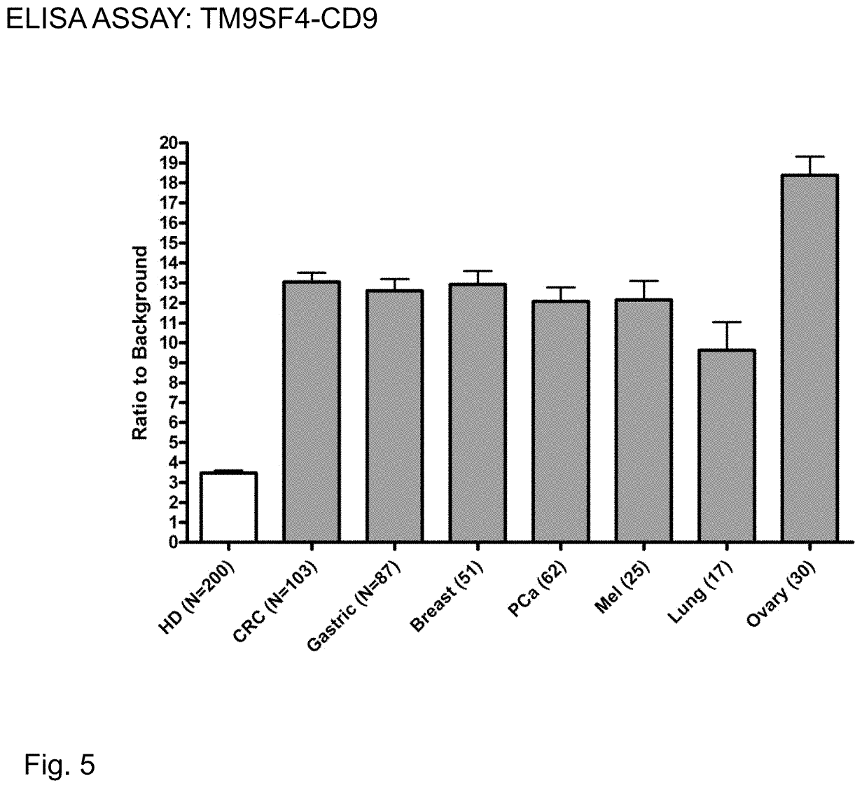

FIG. 5 shows the results of a sandwich ELISA test where 100 μl of pre-cleared (see materials and methods) plasma samples obtained from tumoral patients have been immune-captured through TM9SF4 antibody coated 96 well plates. The detection by CD9 antibody revealed highly specific Ratio to Background values of tumor plasma samples with very low expression in healthy donor plasma samples. In the horizontal axis is reported the tumor group and the number of observations (N). Ratio to Background was calculated by dividing samples adsorbance values for the background value (only PBS in the well Ratio to Background=1).

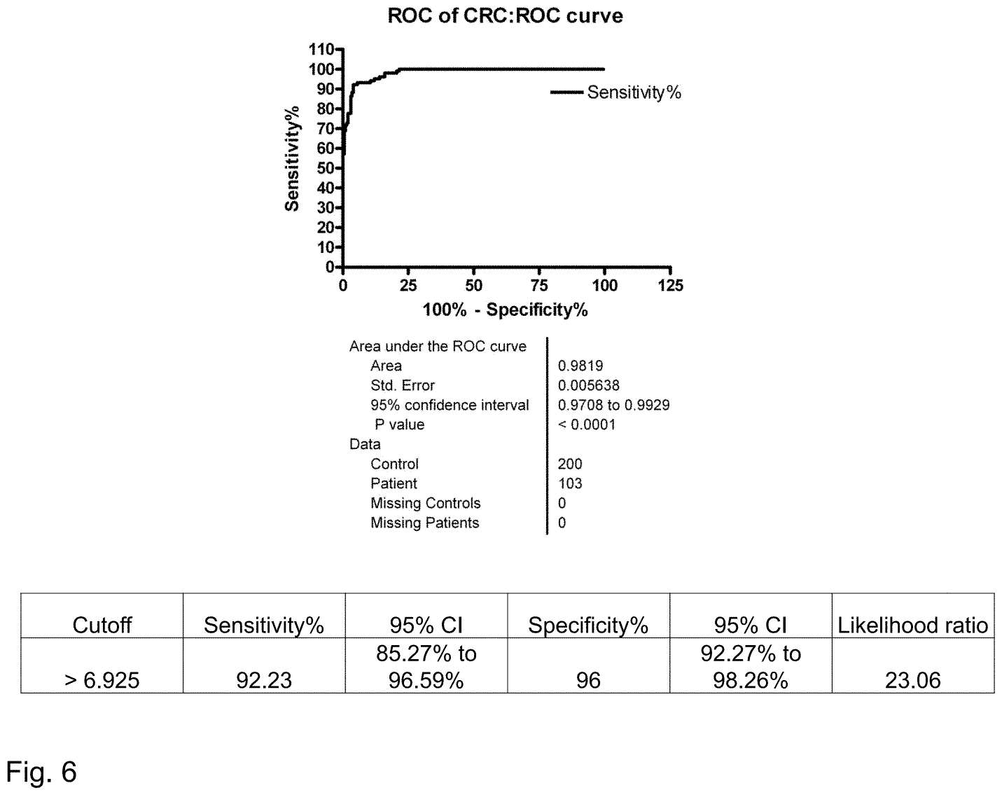

FIG. 6 represents a Receiver Operating Characteristic (ROC) curve calculated by GraphPad Prism program using the Colorectal Cancer (CRC) data reported in FIG. 5. Healthy Donor group was used to calculate the specificity and the optimal threshold of TM9SF4/CD9 ELISA sandwich assay on plasma samples. The figure shows how assuming a threshold of >6.925 the test has a sensitivity >92% and a specificity >95%.

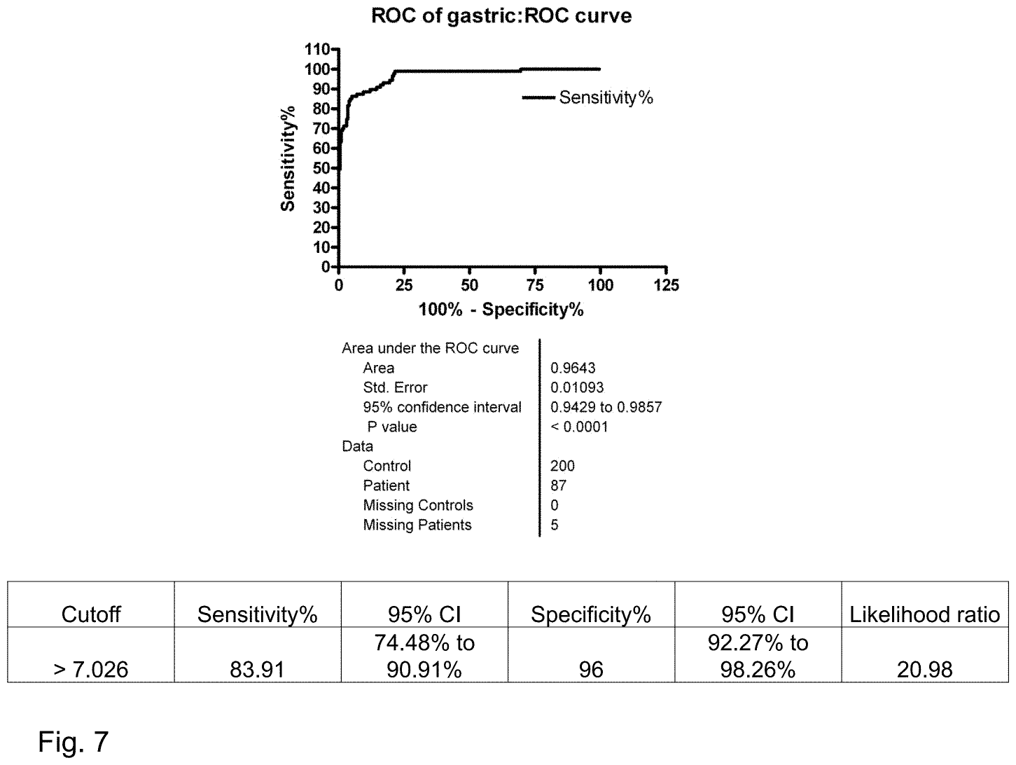

FIG. 7 represents a ROC curve calculated by GraphPad Prism program using the Gastric Cancer data reported in FIG. 5. Healthy Donor group was used to calculate the specificity and the optimal threshold of TM9SF4/CD9 ELISA sandwich assay on plasma samples. The figure shows how assuming a threshold >7.025 the test has a sensitivity >83.9% and a specificity >95%.

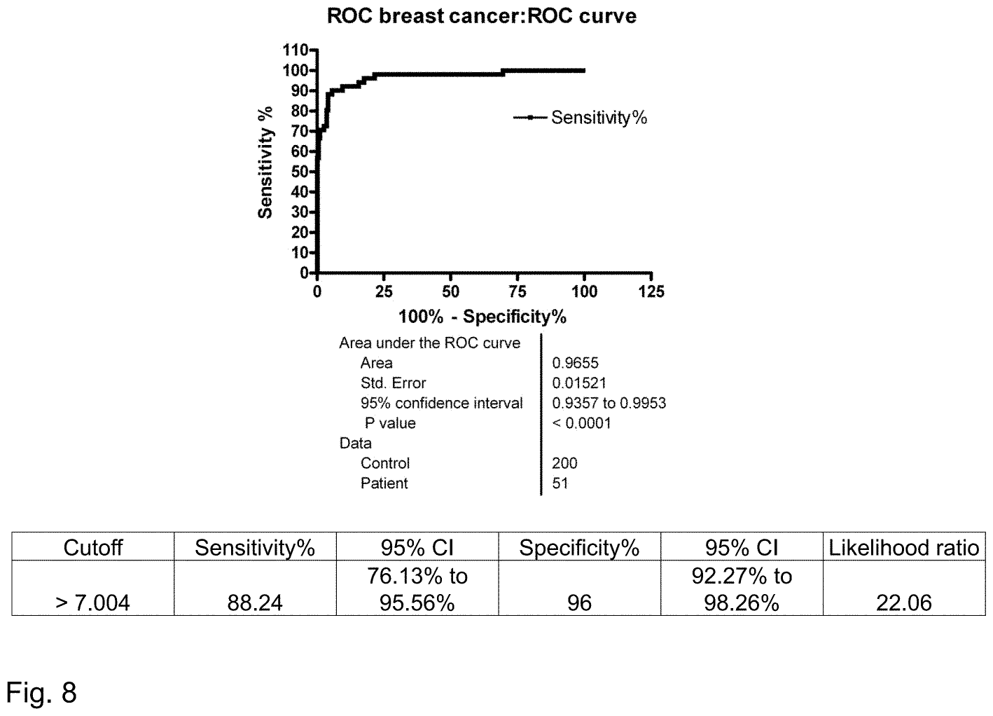

FIG. 8 represents a ROC curve calculated by GraphPad Prism program using the Breast Cancer data reported in FIG. 5. Healthy Donor group was used to calculate the specificity and the optimal threshold of TM9SF4/CD9 ELISA sandwich assay on plasma samples. The figure shows how assuming a threshold >7.004 the test has a sensitivity >88.2% and a specificity >95%.

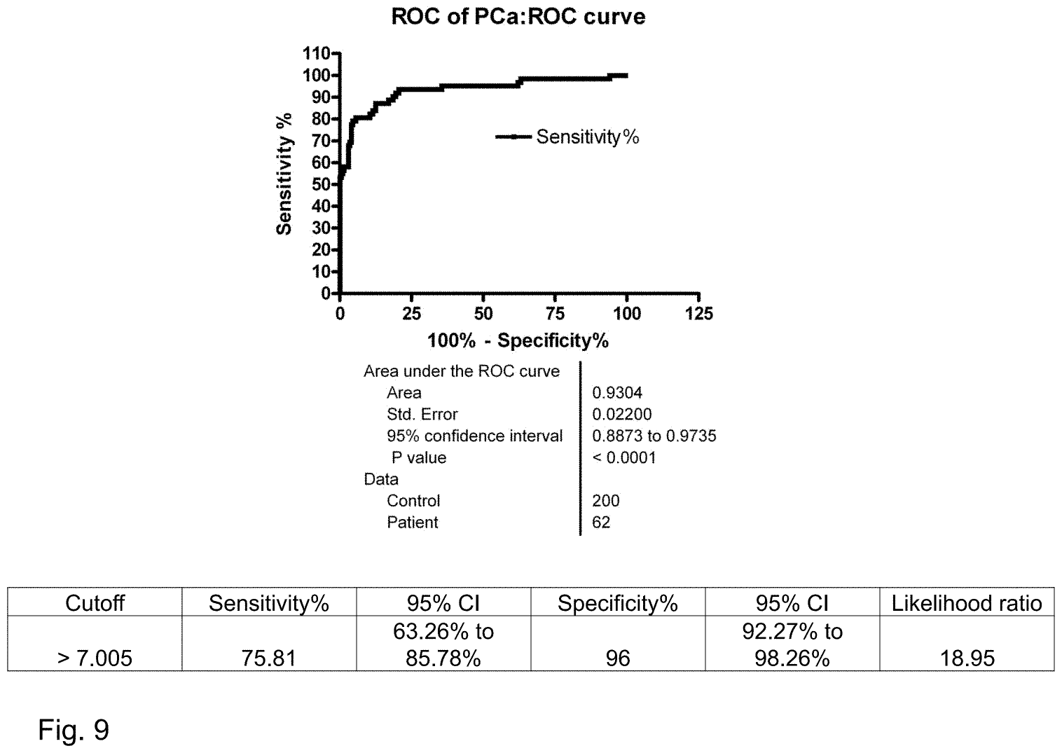

FIG. 9 represents a ROC curve calculated by GraphPad Prism program using the Prostate Cancer data reported in FIG. 5. Healthy Donor group was used to calculate the specificity and the optimal threshold of TM9SF4/CD9 ELISA sandwich assay on plasma samples. figure shows how assuming a threshold >7.005 the test has a sensitivity >75.8% and a specificity >95%.

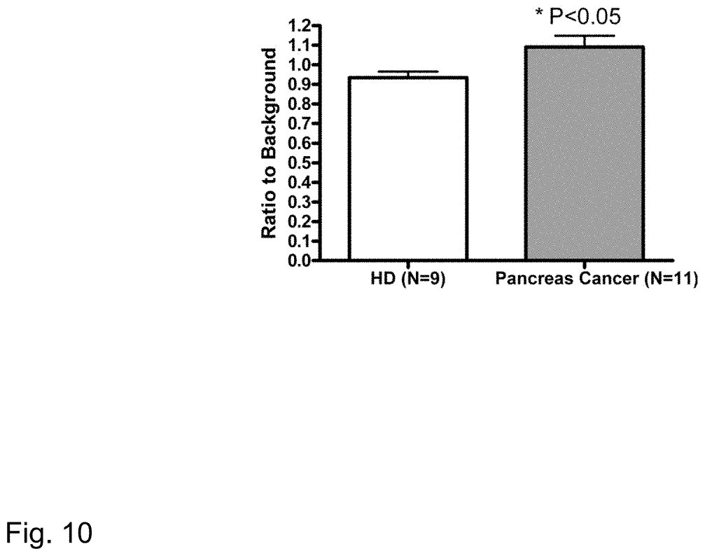

FIG. 10 shows the results of a sandwich ELISA test where 100 μl of pre-cleared (see materials and methods) SERUM samples obtained from tumoral patients have been immune-captured through TM9SF4 antibody coated 96 well plates. The detection by CD9 antibody revealed a significant higher Ratio to Background values of tumor serum samples when compared to healthy donor serum samples. These results suggest that the test ELISA TM9SF4/CD9 is suitable also for Pancreas Cancer plasma samples.

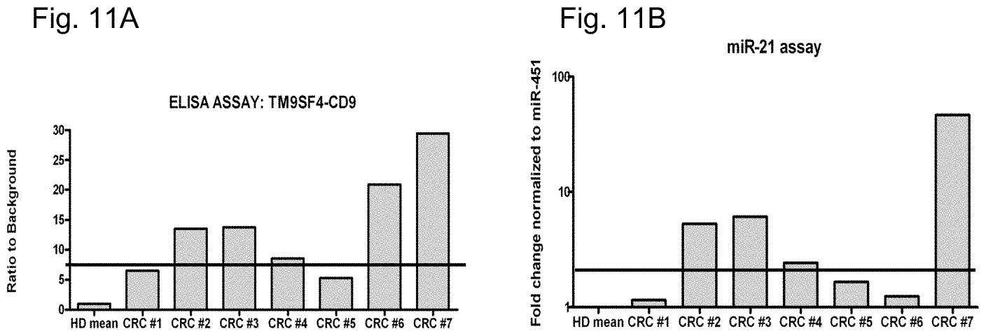

FIG. 11-A shows the results of a sandwich ELISA test where 100 μl of pre-cleared (see materials and methods) plasma samples obtained from seven colorectal cancer (CRC #1-#7) and control group (healthy donors—HD) have been immune-captured through TM9SF4 antibody coated 96 well plates. FIG. 11-B shows the relative expression of extracellular vesicle-(EV)-derived miR-21 (normalized to miR-451) from 100 μl of the SAME set of samples. The TM9SF4-positive vesicles were captured using anti-TM9SF4-antibody-coated beads and RNA was extracted and analysed by RT-qPCR as described in the Material and Methods section. The diagnostic threshold (horizontal line) for the ELISA assay was set as previously described (see materials and methods), and for the miR-21 assay set at a value 2-fold greater than the mean value of the control group. Surprisingly, 6 out of 7 CRC samples showed matched diagnostic results, suggesting a correlation between these two TM9SF4-immunocapture-based assays.

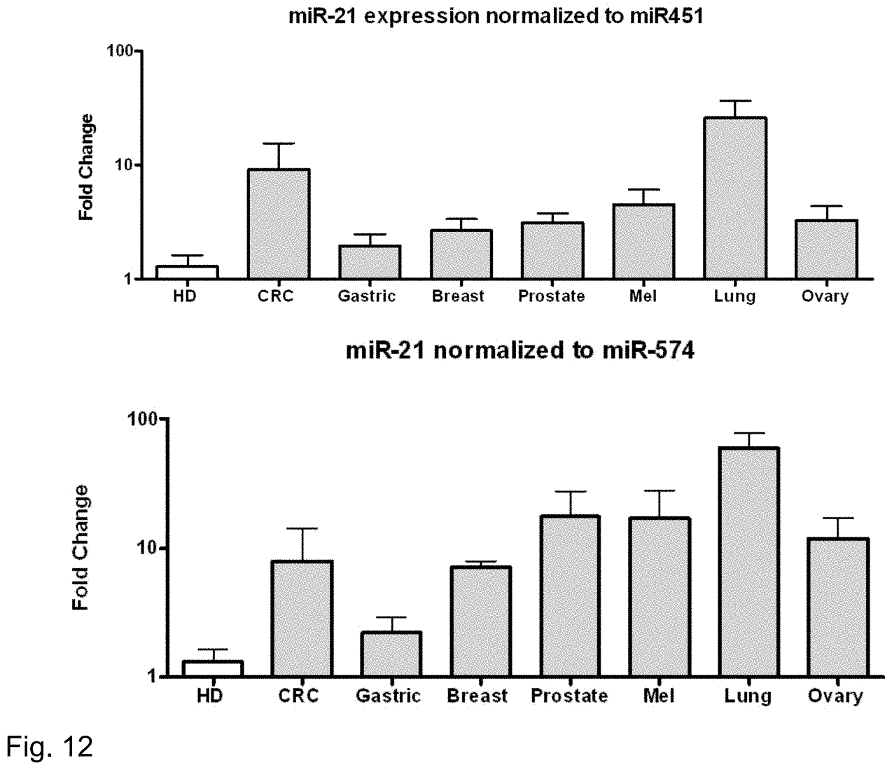

FIG. 12 shows the relative expression of EV-derived miR-21 (normalized to miR-451 or to miR-574) from 100 μl of plasma from cancer patients (Colorectal Cancer (CRC) N=7; Gastric Cancer N=6; Breast Cancer N=6; Prostate Disease N=5; Melanoma N=5; Ovary N=6; Lung Cancer N=6) and control group (healthy donors N=11). The TM9SF4-positive EVs were captured using anti-TM9SF4-antibody-coated beads and RNA was extracted and analyzed by RT-qPCR as described in the Material and Methods section. The data suggest that EV-derived miR-21 is over-expressed in the plasma of cancer patients and that both miR-451 and miR-574 are suitable reference miRNAs for determining the relative expression of tumor-derived miRNAs from EVs.

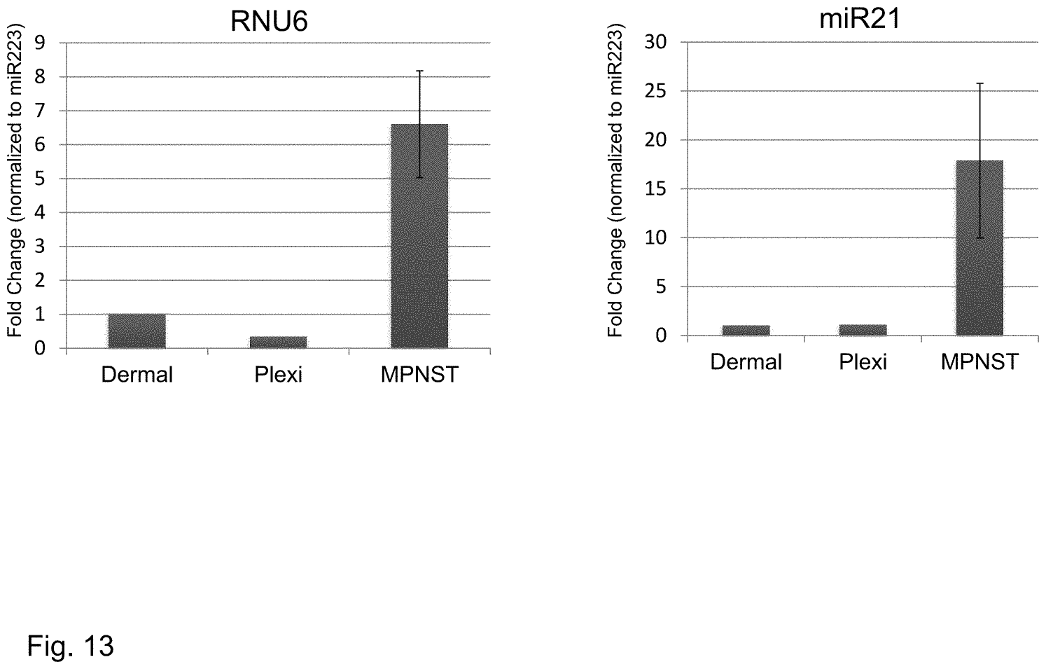

FIG. 13 shows the relative expression of EV-derived RNU6 and EV-derived miR-21 (normalized to miR-223) from 1 ml of concentrated (10×) cell supernatant from dermal, plexiform and MPNST cell lines. The TM9SF4-positive EVs were captured using anti-TM9SF4-antibody-coated beads and RNA was extracted and analyzed by RT-qPCR as described in the Material and Methods section. The data suggest that EV-derived RNU6 and miR-21 are over-expressed in the supernatant of human cancer cell lines (MPNST) but not in the supernatant of benign tumor-derived cell lines (plexiform) or normal cell lines (dermal).

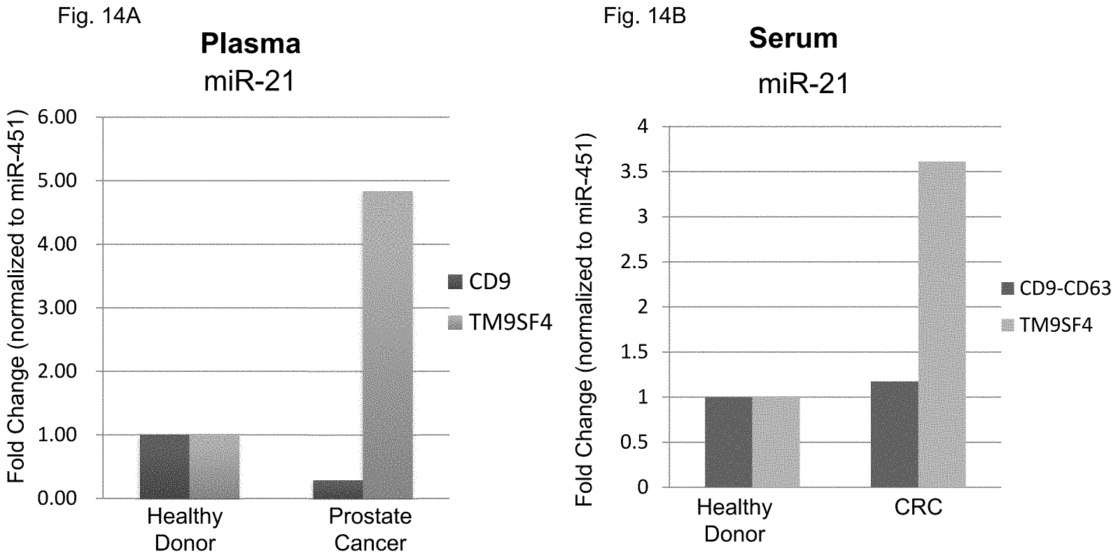

FIG. 14-A shows the relative expression of EV-derived miR-21 (normalized to miR-451) from 100 μl of plasma from a prostate cancer patient and a healthy donor. The EVs were captured using anti-CD9-antibody-coated beads or anti-TM9SF4-antibody-coated beads. RNA was extracted and analysed by RT-qPCR as described in the Material and Methods section. FIG. 14-B shows the relative expression of EV-derived miR-21 (normalized to miR-451) from 100 μl of serum from a colorectal cancer (CRC) patient and a healthy donor. The EVs were captured using beads coated with both anti-CD9 and anti-CD63 antibodies or anti-TM9SF4-antibody-coated beads and RNA was extracted and analyzed by RT-qPCR as described in the Material and Methods section. The data from FIG. 14-A and -B suggest that immunocapture of tumor-derived EVs with anti-TM9SF4-antibody-coated beads enriches for miR-21 (a well-known cancer-associate miRNA) in BOTH plasma and serum. Conversely EV capture with antibodies targeting generic EV-markers (CD9 or CD63) does not enrich for miR-21.

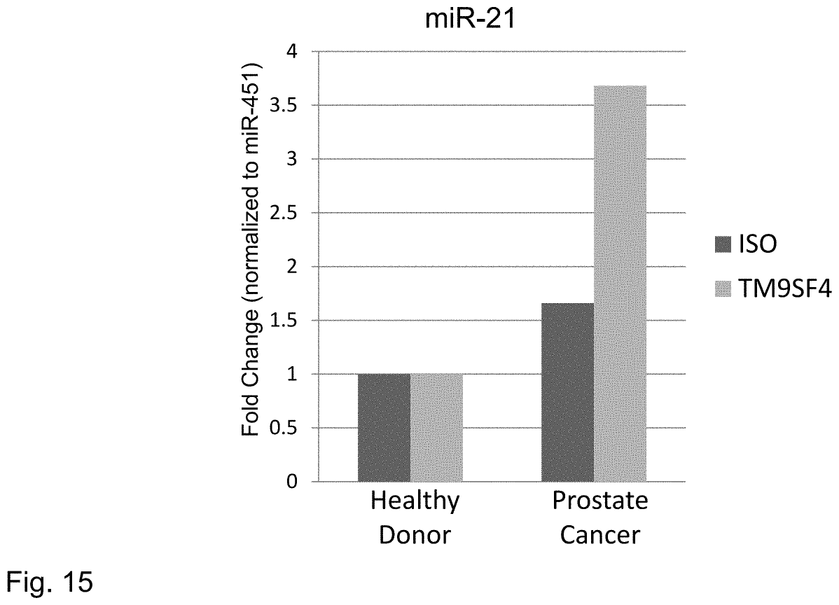

FIG. 15 shows the relative expression of EV-derived miR-21 (normalized to miR-451) from 100 μl of plasma from a prostate cancer patient and a healthy donor. The EVs were captured using anti-TM9SF4-antibody-coated beads or beads coated with isotype-matched-IgG antibodies (ISO) for assessing aspecific binding. RNA was extracted and analyzed by RT-qPCR as described in the Material and Methods section. The data shows the specific enrichment of TM9SF4-positive-EVs using anti-TM9SF4-Ab-coated beads while low aspecific binding was observed in the plasma of the cancer patient.

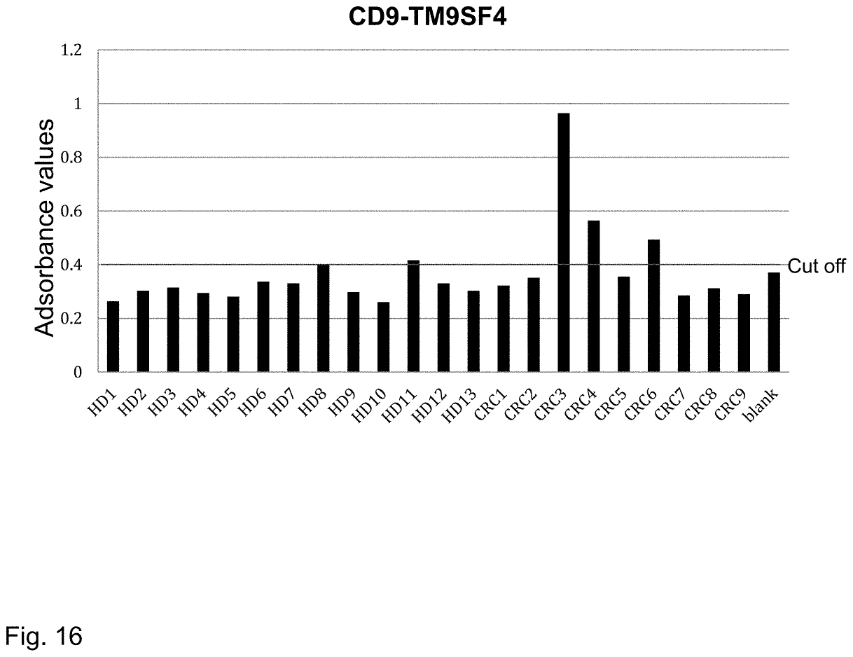

FIG. 16 shows the results of a sandwich ELISA test where 100 μl of pre-cleared (see materials and methods) plasma samples obtained from tumoral patients and healthy donors have been immune-captured through CD9 antibody coated 96 well plates. The detection by TM9SF4 antibody revealed that inverting the capture and detection antibody used in FIG. 5 is not useful to distinguish tumoral origin plasma samples from healthy donor plasma samples. Ratio to Background was calculated by dividing samples adsorbance values for the background value (only PBS in the well Ratio to Background=1).

### Methods

What follows is a description of the methods used in the examples for isolating and analysing the exosomes. The skilled man in the art will recognise that alternative, equivalent, methods exist.

### Exosome Isolation by Ultracentrifugation Protocol

Conditioned medium for exosome preparation and analysis should be collected from 80-90% confluent cells of interest.

Supernatant from cell culture are collected in sterile conditions and added with protease inhibitors diluted 1:1.000, pre-cleared by filtration (0.2 μm), and Ultracentrifuged (ca. 50 mL/tube) at 110.000 g for 1.5 hour at +4° C. The supernatant is then removed and discarded. The pellet is re-suspend in 100 μl of ice cold PBS before dilution in 50 mL ice cold 1×PBS and ultracentrifuged at 110.000 g for 1.5 hour at +4° C. The resulting pellet is re-suspended in 100 μl PBS and vortexed for 30 seconds before pipetting for experimentation.

### Standard Protocol for Protein Markers Detection by FACS Analysis

Exosomal concentration is quantified using Bradford method for protein quantification. Exosomes isolated from cell lines supernatants are incubated at 4° C. over night with aldehyde/sulfate latex beads (4% w/v, 4 μm) in 1:20 ratio. After a washing step in PBS, the exosomes adsorbed on beads surface are incubated in PBS+0.5% BSA with relevant primary antibody (for a final concentration of 5 μg/ml) and kept 1 h at 4° C. Following a washing step with PBS+0.5% BSA, samples are incubated for 45′ at 4° C. with the correspondent secondary antibody (AlexaFluor 488 mouse, rabbit or goat diluted 1:1000). After a final washing step in PBS, samples are resuspended in 300μ1 PBS and analyzed at FACSCalibur (BD). Isotype-matched antibodies or secondary antibodies alone are used as negative control. Median fluorescence intensity of each sample is read using FLI channel and normalized for its negative control.

### Sample Collection

Inclusion criteria comprised only newly diagnosed case of cancer, none of the patients had previously received radio or chemotherapy treatment or underwent surgery before blood collection. All patients gave signed consent before included to the study. The study was conducted by Riga East university Hospital and was approved by a local ethical committee and it was conformed to Declaration of Helsinki. Blood have been collected in 10 ml EDTA tubes, gently inverted and centrifuged at 1500g 10′ RT in 30 minutes from the moment of the blood collection.

Blood center of North Estonia Hospital provided healthy certified donor plasma.

### Immunohistochemical Examination of Tissue

Tissue sections were immunostained to visualize cells that were positive for TM9SF4. Antigen retrieval was achieved by incubation the slides at Tris/EDTA buffer at pH=9.0 at scientific microwave for 30 min. Endogenous peroxidase activity was blocked with 3.0% H₂O₂ for 10 min. Aspecific primary antibody binding was blocked with normal horse serum prior to antibody incubation. The slides were incubated overnight at 4° C. with rabbit polyclonal TM9SF4 antibody (dilution 1:400). The slides were incubated at room temperature for 1 hour at dilution 1:100. Antibodies binding was detected using the EnVision reagent (1 hour at room temperature). The immunoperoxidase reaction colour was developed by incubating the slides with diaminobenzidine for 7 min. A negative control that omitted the primary antibody was included for each experiment.

### Imaging and Quantitation of Cells

For every specimen was given a score according to the intensity of the nucleic or cytoplasmic staining (no staining=0, weak staining=1, moderate staining=2, strong staining=3) and the extent of stained cells (0%=score 0; 1-10%=1; 11-50%=2; 51>=score 3. Negative means 0% area staining. Focally positive means 1-80% area staining, diffusely positive means 81-100% area staining. For Breast, Lung and Melanoma have been counted the number of cancers positive cells/mm2.

### Data Analyses

The results for morphological data were expressed as the means ±SD. Morphological and immunohistochemical data were analysed by two-way ANOVA followed by Bonferroni post test for comparison between the groups. The correlation with clinical and histopathological data was assessed by Spearman test. In all tests, p value of <0.05 was considered statistically significant. SPSS 21. version software was used for the statistical analysis.

### Standard Protocol for Protein Markers Detection by Sandwich ELISA Assay

ELISA assay for purified exosomes by ultracentrifugated conditioned media: 40, 20, 10 and 5 μg/100 μl PBS of isolated exosomes and 100 μl of PBS as negative control (0 μg) are loaded onto a 96 well plate pre-coated with TM9SF4 (2 μg/m1) antibody (transparent plate). Briefly, 96 well plates are pre-coated with the relevant capture antibody, washed thrice with PBS+0.05% TWEEN (washing buffer), added with the isolated exosomes, and incubated overnight at 37° C. After three washes with washing buffer, the plates are incubated with CD9 detection antibody, incubated for 2 hrs at 37° C., washed thrice with washing buffer, incubated for one hour at 37° C. with the corresponded secondary antibody and washed thrice with washing buffer. 100 μl TMB (tetramethylbenzidine) are added to each well and after 5 minutes the reaction is stopped by addition of 100 μl of stop solution (1N sulfuric acid).

The O/D absorbance is read with a M1000 Tecan at 450 nm.

### ELISA Assay for Biological Fluids (Plasma and Serum)

Plasma and serum samples are stored at −80° C., thawed at room temperature and pre-cleared after the addition of 1:500 protease inhibitors cocktail centrifuging at 1200 g 20′ 4° C., transferring the supernatant in another vial and centrifuging again at 10000 g 30′ at 4° C. The supernatant obtained is called pre-cleared and is used for the following analysis.

Briefly 100 μl of pre-cleared plasma or serum are incubated overnight at 4° C. in 96 well plates pre-coated with TM9SF4 antibody (2 μg/ml). After three washes with washing buffer, the plates are incubated with CD9 detection antibody, incubated for 2 hrs at 4° C., washed thrice with washing buffer, incubated for one hour at 4° C. with the corresponded secondary antibody and washed thrice with washing buffer. 100 μl TMB (tetramethylbenzidine) are added to each well and after 5 minutes the reaction is stopped by addition of 100 μl of stop solution (1N sulfuric acid).

The 0/D absorbance is read with a M1000 Tecan at 450 nm.

### Preparation of TM9SF4 Coated Beads

Beads coated with a TM9SF4 antibody can be obtained by using method known to the skilled man in that art or modifications thereof.

### RNA and miRNA Extraction From Immunocaptured Exosomes

### Exosome Isolation by Immunocapture Through TM9SF4 Pre-Coated Beads: Culture Media or Biological Fluids (Plasma and Serum)

10 mL supernatant from cell culture are added with Protease inhibitors diluted at 1:1000 and concentrated 10× using Centrifugal Filter Units (Millipore). 1 ml 10× medium is then incubated overnight at 4° C. with immunobeads pre-coated with TM9SF4 antibody.

Immunocaptured EVs are washed thrice with PBS +Tween 0.01%, and treated with 0.7 ml QIAZOL.

100 μl of pre-cleared plasma or serum are diluted with 900 μl of PBS 1× and incubated overnight at 4° C. in a rotator with 10 μl of TM9SF4 pre-coated beads. Beads are washed thrice with PBS+Tween 0.01% and treated with 0.7 ml QIAZOL.

Total RNA is extracted using Total RNA extraction kit (Hansabiomed) and eluted RNA is quantified at Nanodrop.

### miRNA and snoRNA Amplification and RT-qPCR Analysis

miRNA were retro-transcribed using a miScript II RT Kit (Qiagen) and 0.3 ng cDNA were amplified by qRT-PCR in CFX96™ real-time PCR detection system (BIORAD) with miScript SYBR Green PCR kit (Qiagen), using miScript primer assays (Qiagen) targeting miR-21 (Cat. Num: MS00009079), RNU6, (Cat. Num: MS00033740) and the reference miRNAs, miR-451 (Cat. Num.: MS00004242), miR-574 (Cat. Num.: MS00032025) and miR-223 (Cat. Num.: MS00003871).

### REFERENCES CITED

1. Carroll S L, Ratner N. How does the Schwann cell lineage form tumors in NF1? Glia. 2008; 56(14):1590-605. Epub 2008 Sep. 23. doi: 10.1002/glia.20776. PubMed PMID: 18803326; PubMed Central PMCID: PMC2652636.
2. Evans D G, Baser M E, McGaughran J, Sharif S, Howard E, Moran A. Malignant peripheral nerve sheath tumours in neurofibromatosis 1. J Med Genet. 2002; 39(5):311-4. Epub 2002 May 16. PubMed PMID: 12011145; PubMed Central PMCID: PMC1735122.
3. Korf B R. Malignancy in neurofibromatosis type 1. Oncologist 2000; 5(6):477-85.
4. Lewis J J, Brennan M F. Soft tissue sarcomas. Curr Probl Surg. 1996; 33(10):817-72. Epub 1996 Oct. 1. PubMed PMID: 8885853.
5. Woodruff J M K, H. P.; Louis, D. N.; Scheithauer, B. W. Malignant peripheral nerve sheath tumour (MPNST). In: Kleihues P C, W. K., editor. Pathology and Genetics of Tumours of the Nervous System. First ed. Lyon: IARC Press; 2000. p. 172-4.
6. Ducatman B S, Scheithauer B W, Piepgras D G, Reiman H M, Ilstrup D M. Malignant peripheral nerve sheath tumors. A clinicopathologic study of 120 cases. Cancer. 1986; 57(10):2006-21. Epub 1986 May 15. PubMed PMID: 3082508.
7. Ferner R E, Gutmann D H. International consensus statement on malignant peripheral nerve sheath tumors in neurofibromatosis. Cancer Res. 2002; 62(5):1573-7. Epub 2002 Mar. 16. PubMed PMID: 11894862.
8. McQueen M, MacCollin M, Gusella J, Plotkin S R. Patient and physician attitudes regarding clinical trials in neurofibromatosis 1. J Neurosci Nurs. 2008; 40(6):341-5. Epub 2009 Jan. 28. PubMed PMID: 19170300.
9. Mathivanan S, Ji H, Simpson R J (2010) Exosomes: extracellular organelles important in intercellular communication. J Proteomics 73. 19074920.
10. Keller S, Sanderson M P, Stoeck A, Altevogt P. Exosomes: from biogenesis and secretion to biological function. Immunol Lett. 2006 Nov. 15; 107(2):102-8. Epub 2006 Oct. 17.
11. Simons M, Raposo G. Exosomes-vesicular carriers for intercellular communication. CurrOpinCell Biol. 2009; 21(4):575-81.
12. Simpson R J, Jensen S S, Lim J W. Proteomic profiling of exosomes: current perspectives. Proteomics. 2008 October; 8(19):4083-99. doi: 10.1002/pmic.200800109.
13. Mathivanan S, Lim J W, Tauro B J, Ji H, Moritz R L, Simpson R J. Proteomics analysis of A33 immunoaffinity-purified exosomes released from the human colon tumor cell line LIM1215 reveals a tissue-specific protein signature. MolCell Proteomics. 2010; 9(2): 197-208.
14. Valadi H, Ekstrom K, Bossios A, Sjostrand M, Lee J J, Lotvall J O. Exosome-mediated transfer of mRNAs and microRNAs is a novel mechanism of genetic exchange between cells. NatCell Biol. 2007; 9(6):654-9.
15. Caby M P, Lankar D, Vincendeau-Scherrer C, Raposo G, Bonnerot C. Exosomal-like vesicles are present in human blood plasma. Int Immunol. 2005 July; 17(7):879-87.
16. Mitchell P J, Welton J, Staffurth J, Court J, Mason M D, Tabi Z, Clayton A. Can urinary exosomes act as treatment response markers in prostate cancer? 12; 7:4. doi: 10.1186/1479-5876-7-4.
17. Skog J, Wurdinger T, van Rijn S, Meijer D H, Gainche L, Sena-Esteves M, et al. Glioblastoma microvesicles transport RNA and proteins that promote tumour growth and provide diagnostic biomarkers. NatCell Biol. 2008; 10(12):1470-6.
18. Logozzi M, De Milito A, Lugini L, Borghi M, Calabrò L, Spada M, Perdicchio M, Marino M L, Federici C, Iessi E, Brambilla D, Venturi G, Lozupone F, Santinami M, Huber V, Maio M, Rivoltini L, Fais S. High levels of_exosomes_expressing CD63 and caveolin-1 in plasma of melanoma patients. PLoS One. 2009; 4(4):e5219.
19. Duijvesz D, Luider T, Bangma C H, Jenster G. Exosomes_as biomarker treasure_l chests_for prostate cancer. Eur Urol. 2011 May;
20. Vlassov A V, Magdaleno S, Setterquist R, Conrad R. Exosomes: current knowledge of their composition, biological functions, and diagnostic and therapeutic potentials. Biochim Biophys Acta. 2012 July; 1820(7):940-8.
21. Corrado C, Raimondo S, Chiesi A, Ciccia F, De Leo G, Alessandro R. Exosomes_as intercellular signaling organelles involved in health and disease: basic science and clinical applications. Int J Mol Sci. 2013 Mar. 6; 14(3):5338-66.
22. Chluba-de Tapia J, de Tapia M, Jäggin V, Eberle AN. Cloning of a human multispanning membrane protein cDNA: evidence for a new protein family. Gene. 1997 Sep. 15; 197(1-2):195-204.
23. Lozupone F, Perdicchio M, Brambilla D, Borghi M, Meschini S, Barca S, Marino ML, Logozzi M, Federici C, Iessi E, de Milito A, Fais S. The human homologue of Dictyostelium discoideum phg1A is expressed by human metastatic melanoma cells. EMBO Rep. 2009 December; 10(12):1348-54. doi: 10.1038/embor.2009.236. Epub 2009 Nov. 6.
24. Mackinnon R N, Selan C, Wall M, Baker E, Nandurkar H, Campbell L J. The paradox of 20q11.21 amplification in a subset of cases of myeloid malignancy with chromosome 20 deletion. Genes Chromosomes Cancer. 2010 November; 49(11):998-1013. doi: 10.1002/gcc.20806.
25. Fais S. Proton pump inhibitor-induced tumour cell death by inhibition of a detoxification mechanism. J Intern Med. 2010 May; 267(5):515-25. doi: 10.1111/j.1365-2796.2010.02225.x.
26. Perrin J, Mortier M, Jacomin A C, Viargues P, Thevenon D, Fauvarque M O. The nonaspanins TM9SF2 and TM9SF4 regulate the plasma membrane localization and signalling activity of the peptidoglycan recognition protein PGRP-LC in Drosophila. J Innate Immun. 2015; 7(1):37-46. doi: 10.1159/000365112. Epub 2014 Aug. 13.
27. Caruso R A, Fedele F, Finocchiaro G, Arena G, Venuti A. Neutrophil-tumor cell phagocytosis (cannibalism) in human tumors: an update and literature review. Exp Oncol. 2012 34:306-11.
28. Caruso R A, Muda A O, Bersiga A, Rigoli L, Inferrera C. Morphological evidence of neutrophil-tumor cell phagocytosis (cannibalism) in human gastric adenocarcinomas. Ultrastruct Pathol. 2002 26:315-21.
29. McBurney McBurney M I, Van Soest P J, Jeraci J L. Colonic carcinogenesis: the microbial feast or famine mechanism. Nutr Cancer. 1987; 10(1-2):23-8.
30. Bansal C, Tiwari V, Singh U, Srivastava A, Misra J. Cell Cannibalism: A cytological study in effusion samples. J Cytol. 2011 28:57-60.
31. Lozupone F, Borghi M, Marzoli F, Azzarito T, Matarrese P, lessi E, Venturi G, Meschini S, Canitano A, Bona R, Cara A, Fais S. TM9SF4 is a novel V-ATPase-interacting protein that modulates tumor pH alterations associated with drug resistance and invasiveness of colon cancer cells. Oncogene. 2015 Feb. 9. doi: 10.1038/onc.2014.437. [Epub ahead of print]
32. Ambros V, Lee R C, Lavanway A, Williams P T, Jewell D. MicroRNAs and other tiny endogenous RNAs in C. elegans. Curr Biol. 2003; 13:807-818.
33. Jazbutyte V, Thum T. MicroRNA-21: From cancer to cardiovascular disease. Curr Drug Targets. 2010; 11:926-935.
34. Esquela-Kerscher A, Slack F J. Oncomirs—microRNAs with a role in cancer. Nat Rev Cancer. 2006; 6:259-269. [PubMed]
35. Zhang W, Dahlberg J E, Tam W. MicroRNAs in tumor-igenesis: A primer. Am J Pathol. 2007; 171:728-738. [PMC free article] [PubMed]
36. Chan J A, Krichevsky A M, Kosik K S. MicroRNA-21 is an antiapoptotic factor in human glioblastoma cells. Cancer Res. 2005; 65:6029-6033.
37. Lim Q E, Zhou L, Ho Y K, Too H P. snoU6 and 5S RNAs are not reliable miRNA reference genes in neuronal differentiation. Neuroscience 2011; 199:32-43.

1. A method for determining in vitro the presence of a tumour in a subject, the method comprising:
a) providing a biological sample obtained from the subject,
b) isolating extracellular vesicles from said sample, wherein this step of isolating extracellular vesicles comprises isolating TM9SF4-positive extracellular vesicles through binding to an anti-TM9SF4 antibody,
c) determining from the extracellular vesicles isolated in step b), a level or a presence of biomarker CD9 protein
d) detecting whether the biomarker determined in step c) is over-expressed in the biological sample relative to the biomarker level in healthy donors,
wherein said detecting of the biomarker level of the CD9 protein determined in step c) by at least an approximate 7-fold increase in the subject compared to the level of the CD9 protein in the healthy donors determines the presence of the tumour in the subject.

2. The method according to claim 1, wherein at least a portion of the extracellular vesicles are exosomes.

3. The method according to claim 1, wherein the tumour originates from colon cancer.

4. A method for determining in vitro tumour transformation status in a subject, such method comprising:
a) providing a biological sample obtained from that subject,
b) isolating extracellular vesicles from said sample, wherein this step of isolating extracellular vesicles comprises isolating TM9SF4-positive extracellular vesicles through binding to an anti-TM9SF4 antibody,
c) determining from the extracellular vesicles isolated in step b), a level or a presence of CD9 protein
d) detecting whether the biomarker determined in step c) is over-expressed in the biological sample relative to the biomarker level in healthy donors,
wherein said detecting of the biomarker level of the CD9 protein determined in step c) by at least an approximate 7-fold increase in the subject compared to the level of the CD9 protein in the healthy donors determines the presence of the tumour in the subject.

5. The method according to claim 4, wherein the biological sample of step a) is obtained from a subject affected by a benign tumour.

6. The method according to claim 4, wherein at least a portion of the extracellular vesicles are exosomes.

7. The method according to claim 4, wherein the tumour originates from colon cancer.

8. The method according to claim 4, wherein the tumour transformation status is the transformation to an MPNST or to a colorectal cancer.

9. A kit for use in determining in vitro presence of a tumour or a tumour transformation status in a subject, such kit comprising an anti-TM9SF4 antibody and an anti CD-9 antibody.

10. The kit according to claim 9, wherein the tumour originates from colon cancer.

11. The kit according to claim 9, wherein the tumour transformation status is transformation to an MPNST or to a colorectal cancer.

12. The kit according to claim 9, further comprising instructions for suitable operational parameters in form of a label or separate insert.

13. A method for detecting and quantifying TM9SF4 and CD9 positive exosomes in a subject suspected to have a tumour such method comprising:
a) providing a biological sample obtained from the subject,
b) isolating extracellular vesicles from said sample, wherein this step of isolating extracellular vesicles comprises isolating TM9SF4-positive extracellular vesicles, and
c) determining, from the extracellular vesicles isolated in step b), a level or a presence of a biomarker selected from a group consisting of CD9 protein.

14. A method to verify or to determine an origin of a biological sample, said method comprising steps of:
a) first isolating TM9SF4-positive extracellular vesicles from the sample;
b) from the isolated TM9SF4-positive vesicles detecting CD9-positive extracellular vesicles;
c) measuring expression ratio of TM9SF4 to CD9 from the isolated vesicles expressing both CD9 and TM9SF4; wherein
the origin of the sample being tumoral when the ratio is at least 7.


SEQUENCE LISTING (4 sequences)

SEQ ID NO: 1 - protein, 642 aa, Homo sapiens
MATAMDWLPWSLLLFSLMCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIHWFSIINSVVVVFFLSGILSMIIIRTLRKDIANYNKEDDIEDTMEESGWKLVHGDVFRPPQYPMILSSLLGSGIQLFCMILIVIFVAMLGMLSPSSRGALMTTACFLFMFMGVFGGFSAGRLYRTLKGHRWKKGAFCTATLYPGVVFGICFVLNCFIWGKHSSGAVPFPTMVALLCMWFGISLPLVYLGYYFGFRKQPYDNPVRTNQIPRQIPEQRWYMNRFVGILMAGILPFGAMFIELFFIFSAIWENQFYYLFGFLFLVFIILVVSCSQISIVMVYFQLCAEDYRWWWRNFLVSGGSAFYVLVYAIFYFVNKLDIVEFIPSLLYFGYTALMVLSFWLLTGTIGFYAAYMFVRKIYAAVKID

SEQ ID NO: 2 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 3 - RNA, 22 nt, Homo sapiens
uagcuuaucagacugauguuga

SEQ ID NO: 4 - DNA, 107 bp, Homo sapiens
gtgctcgcttcggcagcacatatactaaaattggaacgatacagagaagattagcatggcccctgcgcaaggatgacacgcaaattcgtgaagcgttccatattttt